(19) United States
(12) Patent Application Publication
(10) Pub. No.: US 2020/0188116 A1
(43) Pub. Date: Jun. 18, 2020

(54) IMPROVED ABSORBABLE AND BIOCOMPATIBLE GRAFT FOR IMPLANTATION FOLLOWING UPON EXERESIS OF THE IPP PLAQUE

(71) Applicants: Bianca DORDONI, Cremona (IT); Bianca SAMBUSSETI, Cremona (IT); Elisa SAMBUSSETI, Cremona (IT)

(72) Inventor: Antonio SAMBUSSETI, Cremona (IT)

(21) Appl. No.: 16/609,133

(22) PCT Filed: Apr. 24, 2018
(86) PCT No.: PCT/EP2018/060440

§ 371 Date: Oct. 28, 2019

(30) Foreign Application Priority Data
Apr. 28, 2017 (IT) 102017000046258

(51) Int. Cl. A61F 2/26; A61F 2/00

(52) U.S. Cl. CPC A61F 2/26; A61F 2/0063; A61F 2250/0036; A61F 2230/0019; A61F 2230/0021; A61F 2210/0004

(57) ABSTRACT

Described herein is a graft for implantation following upon exeresis of the IPP plaque resulting from Peyronie's disease, the graft including a fabric deriving from fibres of an absorbable polymer chosen from between PGA and PLA, wherein the graft presents a peripheral boundary region which is continuous around the sides of the graft and having a greater thickness than the remaining part of the graft to form a sort of frame, the peripheral boundary region being integral with the remaining part of the graft so as to form a single piece.

### BACKGROUND OF THE INVENTION

### Field of the Invention

The present invention relates to an improved absorbable graft made of biocompatible material such as PGA or PLA to be used in the urological field, in particular as dermal implantation in the exeresis of the IPP (induratio penis plastica) plaque resulting from Peyronie's disease.

Peyronie's disease, for reasons as yet not well known, regards the male genital organ and causes a more or less accentuated penile deformity due to the presence of one or more hard fibrous plaques, with nodular appearance, localised on the corpora cavernosa of the penis, in particular at the level of the tunica albuginea (the sheath that coats the corpora cavernosa of the penis and that represents the supporting structure that determines rigidity of the penis in erection) with irreversible degeneration of the albugineous elastic component.

The area of fibrosis, generically defined as “plaque”, causes a curvature of the penis towards the diseased side. This disease is associated with severe pain and erectile dysfunction in so far as the disappearance of the elastic fibres that occurs in favour of the growth of the dense fibrous tissue of the plaque constitutes an alteration of the mechanical characteristics of the corpora cavernosa.

When the disease has stabilised (for at least six months) and is such as to jeopardise sexual function, it is necessary to resort to surgical therapy, such as surgery of the tunica albuginea or complete exeresis of the plaque.

### Description of the Related Art

The current tendency is to perform a complete exeresis (surgical excision or removal) of the plaque. In this case, the removed section is consequently substituted with a graft or dermal implantation constituted by autologous tissue (i.e., tissue of the subject, such as derma of the patient's thigh) in so far as a tissue is required that will undergo a natural histo-transformation, maintaining the elastic fibres contained therein, to enable renewal of functionality of the organ. In effect, in tissue-repair processes, there is no production of elastic tissue, but only of fibrous tissue, since in the cellular matrix there exist only fibroblasts and not elastoblasts.

Implants, also referred to as “grafts”, enable renewal of the primitive length on the side involved in the cicatricial retraction that shortens the penis, at the same time functioning as support for the autologous tissue of the patient that will come to form in time over said graft.

Synthetic implants, in the corporoplastic technique referred to above, are not today much used in so far as their physiopathological characteristics of engraftment do not enable reconstruction of a symmetrical and congruent tunica albuginea.

It is in fact known that their mechanism of engraftment does not envisage vascular inosculation but only their fibroblastic encapsulation: this would lead to a structure of substitution of the tunica albuginea that is without elastic fibres and is hence not extensible and to a fibrous reaction of the erectile tissue beneath the graft with a tendency to retraction.

In the cases of serious erectile deficit, another type of surgical technique is adopted that envisages the use of penile prostheses of various types, soft, malleable, hydraulic ones, etc., in order to straighten and/or lengthen the organ and re-acquire erection, in combination with autologous dermal grafts. However, this technique entails a high percentage of infectious complications when dermal grafts of an autologous type are used, due to the presence of staphylococci.

Also the use of heterologous grafts (SIS) or synthetic grafts (GORETEX), albeit preventing the aforesaid complications, do not guarantee a high elasticity on account of their tendency towards retraction and of the quality of the reconstruction tissue around them.

Another surgical technique consists in a surgery of resection/incision of the plaque, exeresis of the tunica albuginea and of the corpora cavernosa, and implantation of a graft, preferably autologous coming from the saphenous vein, between the tunica albuginea and the corpora cavernosa. Also in this case, according to the type of graft, the drawbacks set forth above may arise.

Consequently, it is highly desirable to have available implants, in particular synthetic implants, that will maximise the elasticity characteristics, almost as much as the original tissue, and will determine a better quality of the tissue reconstruction with the creation of elastoblasts in addition to the fibroblasts without granulomas, keloids, and the like, by minimising the reaction of retraction to the implant, and the infectious reactions.

Moreover, it is highly desirable to have available implants as described above that are also completely absorbable but that will not detach from the sutured area during absorption of the graft so as to guarantee a symmetrical and congruent tissue reconstruction, and hence an improved reconstruction of the cellular neotissue of the tunica albuginea.

### SUMMARY OF THE INVENTION

The aim of the present invention is to provide an improved graft for implantation following upon exeresis of the IPP plaque that will overcome the drawbacks of the known art, and in particular to provide a biocompatible, highly elastic graft that shows a reduced formation of fibrotic capsule around it, even when it is used to substitute an extensive IPP plaque, and that can be used without giving rise to infections.

Another aim of the present invention is to provide said graft that will also be easy to produce, with the smallest possible number of production steps, and will be economically advantageous, as well as being reliable, functional, and absorbable in order to prevent a subsequent intervention for removal of the graft.

A further aim of the present invention is to provide said graft that will have characteristics such as to guarantee an improved reconstruction of the cellular neotissue, in particular a symmetrical and congruent tissue reconstruction.

The above aims are achieved by the improved absorbable graft according to the invention made of biocompatible material constituted by PGA or PLA that presents the characteristics specified in the annexed independent Claim 1.

Advantageous embodiments of the invention will emerge clearly from the dependent claims.

According to the invention, the graft for implantation following upon exeresis of the IPP (induratio penis plastica) plaque resulting from Peyronie's disease is made of a fabric formed by a single material constituted by a biocompatible and absorbable homopolymer chosen from between PGA and PLA, where the peripheral boundary region of the fabric of the graft has an increased thickness with respect to the remaining part of the fabric of the graft so as to be thickened in a perimetral edge with respect to the remaining part (i.e., the non-perimetral part) of the graft.

The peripheral boundary region is hence continuous in so far as it is set around the perimeter of the graft and is also integral with the remaining part of the graft so as to form a single piece: in effect, the peripheral boundary region and the remaining part of the graft are elements that together constitute a single piece, and that hence cannot be removed/separated from one another.

### BRIEF DESCRIPTION OF THE DRAWINGS

Further characteristics of the invention will emerge more clearly from the ensuing detailed description, with reference to some embodiments thereof provided purely by way of non-limiting example, illustrated in the annexed drawings, wherein:

FIG. 1a is a top plan view of an embodiment of the graft according to the invention;

FIG. 1b is a side view of the graft illustrated in FIG. 1a;

FIG. 1c is a cross-sectional view, partially interrupted, of the graft of FIG. 1a taken along the line 1c-1c;

FIGS. 2a-2c are views that represent possible positioning of the graft of FIG. 1a on a male genital organ in a ventral position, a dorsal position, and a lateral position, respectively; and

FIG. 3 is a cross-sectional view of a male genital organ that has the IPP plaque.

### DESCRIPTION OF THE PREFERRED EMBODIMENTS

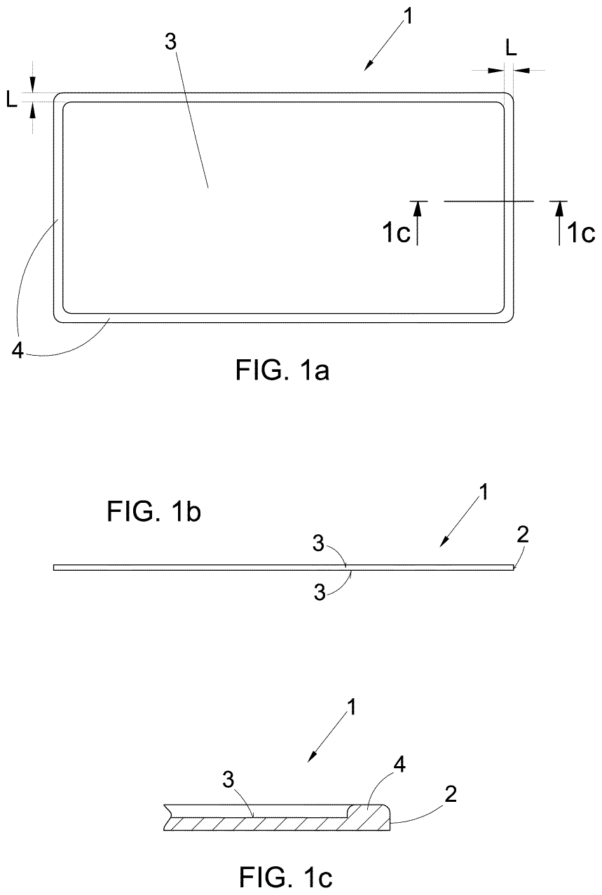

The graft 1 (FIGS. 1a and 1b) according to the present invention has in general a plane, rectangular or square shaped, with dimensions variable according to the possible overall dimensions of the plaque, and optionally with rounded corners.

A dimensional example of said graft is 5 cm×10 cm.

The above graft 1 has a peripheral boundary region 4 that has a greater thickness than the remaining part of the graft so as to present a continuous thickened edge to form a sort of continuous frame (FIG. 1c).

In particular, the thickness of said continuous peripheral boundary region 4 is at least twice the thickness of the remaining part of the graft, even though it may be more than twice as much, without thereby departing from the scope of the present invention.

More preferably, the thickness of said peripheral boundary region 4 is greater than twice the thickness of said remaining part 3.

The edge 4 thus thickened has a width L (FIG. 1a) that is generally approximately 2 to 3 mm from the sides of the graft 1 to the centre thereof.

Said graft 1 is in the form of fabric 2 (with holes) and its thickness (understood as thickness of the non-perimetral part 4) is, for example, around 600 μm.

In the case where the graft 1 is made of PLA, it is preferable for it to be in the form of fabric 2 deriving from an ultralight PLA monofilament.

By PLA it is intended to identify all polymers or copolymers based upon lactic acid (L-lactic acid, D-lactic acid, racemic lactic acid, dimer lactic acid, lactic-acid esters, etc., or a combination thereof). Particularly preferred are fibres of (homopolymer) polylactic acid (PLA).

The above PLA fabric 2 is preferably from 30 and 160 deniers fabric.

In a preferred embodiment, the fabric of the present graft 1 is constituted by fibres of a homopolymer chosen from between the PGA homopolymer and the PLA homopolymer.

When the material of the graft 1 is PGA, the graft is preferably in the form of fabric made of a yarn or of an ultralight monofilament deriving from fibres of PGA (polyglycolide or polyglycol acid), preferably homopolymer.

In addition, said graft can also be provided with one or more reinforcement strips applied and arranged on at least one of the surfaces of the remaining part 3 (that do not include the area of the perimetral edge 4), which are made of the same type of fabric as the PGA graft.

The fabric 2 of the PGA graft 1 including said peripheral boundary region 4 is a single piece.

The increased thickness of the edge 4 (i.e., perimetral region) is directly obtained during manufacture of the fabric: this entails advantages as compared to a bending-back of the end edges in so far as it enables thicknesses to be obtained that are greater than twice the thickness of the remaining part 3, whereas this is not possible in the case of bending-back of the edge.

Furthermore, in the case of bending back of the edge, it is necessary to fix the bent-back part to the rest of the graft with a series of seams, whereas in the present graft no step of sewing of the edge to the remaining part of the graft is envisaged, thus simplifying the production process of the present graft.

The present fabric 2 including the thickened edge 4 can be obtained by interweaving in various ways said PGA monofilament, giving rise to a knit fabric, a woven fabric, or else a non-woven fabric.

It is preferable to use a warp-knit fabric. Thanks to its mechanical consistency, the present graft 1 can be used alone without any need for it to be supported.

When a PGA monofilament is used, the fabric 2 preferably has a linear mass density or grammage between 240 and 320 deniers and the monofilament PGA yarn preferably ranges between 120 and 160 deniers.

When the graft 1 of PGA is constituted by a yarn of PGA fibres, this is a multifilament yarn of 75 deniers/30 filaments (parallel to one another) and is a warp-knit fabric. This embodiment enables the graft 1 to present a greater stiffness in respect to a fabric obtained with monofilament.

Production of the graft 1 in the form of a fabric is carried out in an environment with controlled contamination, in a white chamber, and with low humidity (in the case of PLA). Once production is completed, the graft 1 is put in a double blister pack closed with a sheet of Tyvek to prevent any contamination, and sent on to a gamma-ray sterilisation cycle. At this point, the graft is ready to be used for surgery.

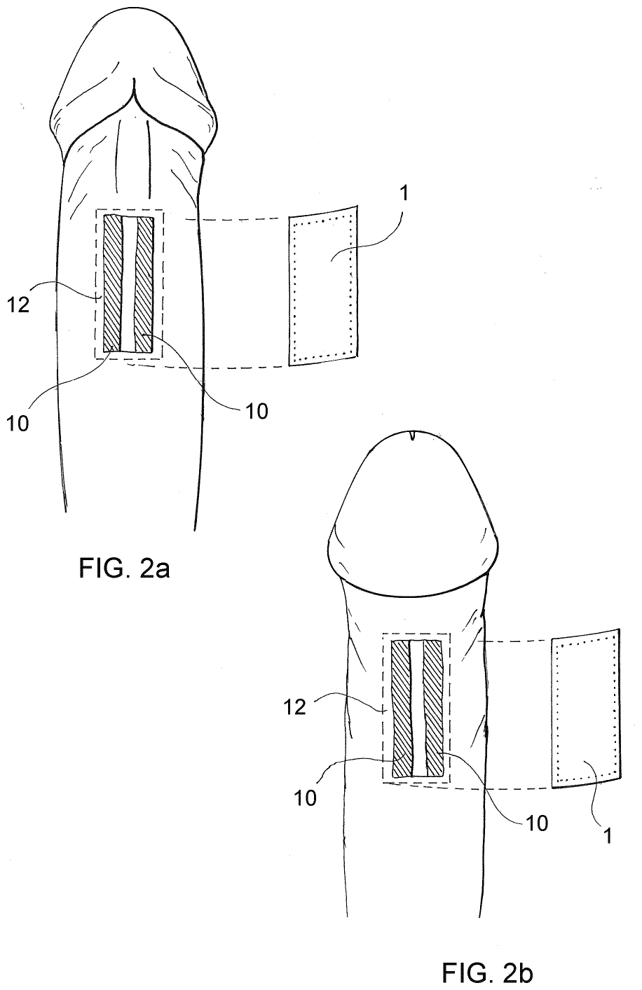

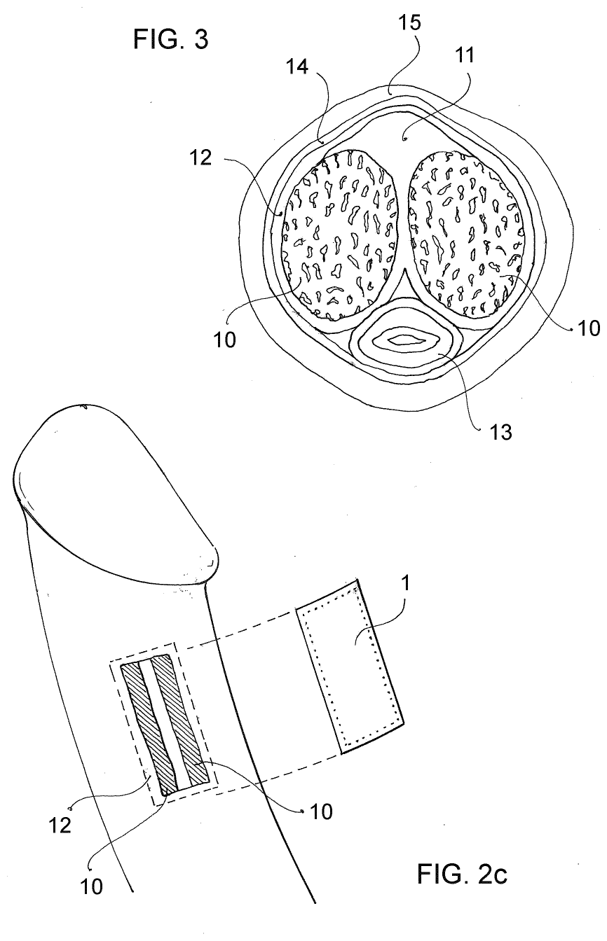

The graft 1 according to the invention is designed to be set on the corpora cavernosa 10 (FIGS. 2a-2c, 3) after removal of the IPP plaque 11 (FIG. 3) and to be sutured in the proximity of the tunica albuginea 12, after cutting of the derma 15 (FIG. 3) and of the Buck's fascia 14 (FIG. 3) has been carried out according to known surgical techniques.

The graft 1 can be sutured to the margins of the cut using thread for sutures with 3/0 or 4/0 thread size, made of absorbable material. Preferably, the thread for suturing the graft is of the same material as the one used for the fabric of the graft.

The main advantage of a graft made of PLA or else PGA is its absorbability during regeneration of the area excised, and consequently its removal is not necessary, as instead occurs for silicone grafts coated with turbostratic carbon. Moreover, it does not give rise to risks of infection, and the re-epithelization quality is high.

Moreover, the above PLA or PGA graft does not present any risk of adherence of the fibrotic capsule to the graft in so far as it is completely absorbable within 3 to 6 months for PLA and within 1 to 2 months for PGA according to the metabolism.

In addition, PGA and PLA present the advantage of being absorbed, giving way to an autologous neotissue that is as elastic as the original one.

It is moreover to be noted that the continuous thickened edge 4 enables more efficient suturing of the graft 1 as compared to conventional grafts made of the same material but with one and the same thickness in each point of the graft. In fact, from clinical studies performed by the present applicant it has been found that the sutures on the edge of a graft with constant thickness that is also the same in every point, including the edge, are too weak and tend to open, causing detachment of the graft from its seat prior to complete absorption thereof.

Instead, the present graft that has the thickened edge 4 has proven better not only in terms of resistance of the sutures, but also in terms of detachment.

In fact, during the aforesaid studies, it has been noted that the central part 3 of the present graft 1 is absorbed faster than the thickened edge 4 so that a detachment of the graft 1 is impossible when the central part 3 of the graft has not yet been completely absorbed.

Consequently, the present graft 1 enables a better reconstruction of the cellular neotissue of the tunica albuginea as compared to conventional grafts in so far as it enables a more symmetrical and congruent reconstruction.

What has just been said applies also in the case of known conventional grafts that have a discontinuous thickened edge, e.g., formed only on some portions of the sides of the graft, and/or that have a edge that is not integral with the remaining part of the fabric, e.g., portions of material fixed in different ways to one or more sides of the graft so as to thicken some portions of each side of the graft.

The present embodiments of the invention may undergo numerous variations and modifications in its details, all within the reach of a person skilled in the branch, that in any case fall within the scope of the invention as specified in the annexed claims.

1-10. (canceled)

11. A graft (1) for implantation, following upon exeresis of the IPP (induratio penis plastica) plaque resulting from Peyronie's disease, comprising a fabric (2) deriving from fibres of an absorbable polymer chosen from between PGA (polyglycolide) and PLA (polylactic acid),
said graft comprising a peripheral boundary region (4) and being wherein said peripheral boundary region (4) is continuous around the sides of the graft and has a greater thickness than the remaining part (3) of said graft (1) to form a sort of continuous frame,
said peripheral boundary region (4) and the remaining part (3) of the graft being elements that together constitute a single piece, and that cannot be removed/separated from one another.

12. The graft (1) according to claim 11, wherein the thickness of said remaining part (3) of the fabric (2) is approximately 600 μm.

13. The graft (1) according to claim 11, wherein the shape is planar, rectangular or squared.

14. The graft (1) according to claim 11, wherein the sutures of said graft to the margins of the cut is performed using thread for sutures with a 3/0 or 4/0 thread size, made of absorbable material, for example PGA, or else of non-absorbable material.

15. The graft (1) according to claim 11, wherein the fabric (2) is made of PLA, derives from an ultralight monofilament and is from 30 to 160 deniers fabric.

16. The graft (1) according to claim 11, wherein the fabric (2) made of PGA including said peripheral boundary region (4) is made of a yarn or of an ultralight monofilament and is in the form of a knit fabric, a woven fabric or else a non-woven fabric.

17. The graft (1) according to claim 11, wherein when a PGA monofilament is used, the fabric (2) has a titre between 240 and 320 deniers and the PGA monofilament is from 120 to 160 deniers.

18. The graft (1) according to claim 11, wherein when a PGA yarn is used, it is a multifilament yarn of 75 deniers/30 filaments.

19. The graft (1) according to claim 11, wherein the fabric (2) made of PGA is provided with one or more reinforcement strips on at least one of its surfaces that are made of the same type of fabric of said PGA graft.

20. The graft (1) according to claim 11, wherein said peripheral boundary region (4) has a width (L) of approximately 2 to 3 mm from the sides of the graft (1) to the centre thereof.

21. The graft of claim 11, wherein the absorbable polymer is a homopolymer.

22. The graft of claim 21, wherein said thickness of said region (4) is at least twice the thickness of said remaining part (3) of said graft (1).

23. The graft of claim 21, wherein said thickness of said region (4) is greater than twice the thickness of said remaining part (3) of said graft (1).

24. The graft (1) according to claim 12, wherein the shape is planar, rectangular or squared.

25. The graft (1) according to claim 13, wherein the shape is planar and rectangular, with dimensions of 5 cm×10 cm.

26. The graft (1) according to claim 11, wherein the fabric (2) made of PGA including said peripheral boundary region (4) is made of a yarn or of an ultralight monofilament and is in the form of a warp knit.

27. The graft (1) according to claim 12, wherein the sutures of said graft to the margins of the cut is performed using thread for sutures with a 3/0 or 4/0 thread size, made of absorbable material, for example PGA, or else of non-absorbable material.

28. The graft (1) according to claim 13, wherein the sutures of said graft to the margins of the cut is performed using thread for sutures with a 3/0 or 4/0 thread size, made of absorbable material, for example PGA, or else of non-absorbable material.

29. The graft (1) according to claim 12, wherein the fabric (2) is made of PLA, derives from an ultralight monofilament and is from 30 to 160 deniers fabric.

30. The graft (1) according to claim 13, wherein the fabric (2) is made of PLA, derives from an ultralight monofilament and is from 30 to 160 deniers fabric.
